(19) United States
(12) Patent Application Publication
(10) Pub. No.: US 2014/0038280 A1
(43) Pub. Date: Feb. 6, 2014

(54) MICROCHIP FOR NUCLIEIC ACID AMPLIFICATION REACTION

(71) Applicant: Sony Corporation, Tokyo (JP)

(72) Inventors: Masaki Sato, Tokyo (JP); Tomohiko Nakamura, Tokyo (JP); Kenzo Machida, Kanagawa (JP); Toshio Watanabe, Kanagawa (JP); Yoshiaki Kato, Gunma (JP); Michihiro Ohnishi, Kanagawa (JP)

(73) Assignee: Sony Corporation, Tokyo (JP)

(21) Appl. No.: 13/953,914

(22) Filed: Jul. 30, 2013

(30) Foreign Application Priority Data
Aug. 6, 2012 (JP) 2012-174415

(51) Int. Cl. C12Q 1/68

(52) U.S. Cl. CPC C12Q 1/6844
USPC 435/302.1; 435/289.1

(57) ABSTRACT

There is provided a microchip for a nucleic acid amplification reaction including a reagent accommodation region including a solid-phase reagent and a particle, the solid-phase reagent containing a substance necessary for a nucleic acid amplification reaction.

### BACKGROUND

The present technology relates to a microchip for a nucleic acid amplification reaction, and more particularly relates to a microchip for a nucleic acid amplification reaction including a solid-phase reagent and a particle, the reagent containing a substance necessary for the nucleic acid amplification reaction.

In recent years, by applying a microfabrication technique in the semiconductor industry, microchips having wells and flow channels for performing chemical and biological analyses formed in a substrate made of silicon or glass have been developed. These microchips have begun to be used, for example, for an electrochemical detector of liquid chromatography or a small-sized electrochemical sensor in medical practice.

An analysis system using such microchips is referred to as a μ-TAS (micro-Total-Analysis System), a lab-on-a-chip, a biochip, or the like, and attracts attention as a technology enabling speed-up, efficiency enhancement, and integration of the chemical and biological analyses, or downsizing of analysis equipment. Since the μ-TAS enables an analysis of a small amount of a sample and disposable use (single-use) of a microchip, application of the μ-TAS to the biological analysis handling a trace amount of a particularly precious sample or many test bodies is expected.

An example of the application of the μ-TAS is a photodetector which introduces substances into a plurality of areas provided in a microchip and chemically detect the substances. An example of the photodetector is a reactor (for example, a real time PCR apparatus) which progresses a reaction such as a nucleic acid amplification reaction of a plurality of substances in wells of the microchip and optically detects a generated substance.

In related art, a microchip-type nucleic-acid amplification apparatus employs a method by which substances necessary for a nucleic acid amplification reaction and a template nucleic acid are all mixed together in advance to prepare a reaction mixture and then the reaction mixture is introduced into a plurality of wells provided in the microchip. In the method, however, there is an issue that it takes labor to prepare the reaction mixture and takes a certain time to introduce the reaction mixture into the wells, so that the nucleic acid amplification reaction progresses in the reaction mixture in the meantime. Accordingly, it is not possible to strictly control a time period of the nucleic acid amplification reaction.

In light of the foregoing, JP 2011-160728A, for example, discloses the microchip for a nucleic acid amplification reaction including a plurality of reagents necessary for a nucleic acid amplification reaction which are laminated and anchored in wells in a predetermined order.

### SUMMARY

The microchip for a nucleic acid amplification reaction described above makes it possible to start the nucleic acid amplification reaction by introducing, into the wells, remaining substances necessary for the nucleic acid amplification reaction and a sample containing a nucleic acid to be amplified, thus making it possible to perform an analysis which is simple and in which a reaction time period of the nucleic acid amplification reaction is controlled. However, in some cases, the analysis is started in a state where the sample introduced into the wells and the anchored reagents are not fully mixed together. Hence, it is desirable to provide a microchip for a nucleic acid amplification reaction in which a sample introduced into the microchip and a solid-phase reagent are easily mixed together.

According to an embodiment of the present technology, there is provided a microchip for a nucleic acid amplification reaction including a reagent accommodation region including a solid-phase reagent and a particle, the solid-phase reagent containing a substance necessary for a nucleic acid amplification reaction.

A particle accommodation section allowing the particle to be accommodated therein may be provided in the reagent accommodation region at a predetermined position.

The particle may have a higher specific gravity than a sample to be introduced into the reagent accommodation region, and the particle accommodation section may be a recessed portion of a bottom surface of the reagent accommodation region.

The bottom surface of the reagent accommodation region may be formed as a curved surface bulging toward a surface facing the bottom surface.

The particle may have a lower specific gravity than a sample to be introduced into the reagent accommodation region, and the particle accommodation section may be a recessed portion of a surface facing a bottom surface of the reagent accommodation region.

The surface facing the bottom surface of the reagent accommodation region may be formed as a curved surface bulging toward the bottom surface.

The particle may be magnetic, and a magnetic member may be provided at a position from which magnetic force is exerted on the particle accommodation section.

The particle may be thermally melted.

Further, the reagent accommodation region may be a reaction site of the nucleic acid amplification reaction.

The microchip for a nucleic acid amplification reaction may include a reaction site for the nucleic acid amplification reaction which is connected with the reagent accommodation region by a flow channel, and the reagent accommodation region may be connected with an introduction section for a sample by a flow channel.

According to the embodiments of the present technology, there is provided a microchip for a nucleic acid amplification reaction in which a sample introduced into the microchip and a solid-phase reagent are easily mixed together.

### BRIEF DESCRIPTION OF THE DRAWINGS

FIG. 1 is a schematic diagram of a microchip 1a for a nucleic acid amplification reaction according to a first embodiment of the present technology, FIG. 1A illustrating a top diagram of the microchip 1a for a nucleic acid amplification reaction, FIG. 1B illustrating a cross-sectional diagram of the microchip 1a for a nucleic acid amplification reaction taken along the P-P line of FIG. 1A;

FIG. 2 is a schematic cross-sectional diagram illustrating a structure of a reaction region 43a of the microchip 1a;

FIG. 3 is a schematic cross-sectional diagram illustrating a structure of a modification of the microchip 1a;

FIG. 4 is a schematic cross-sectional diagram illustrating a structure of a reaction region 43b of a microchip 1b for a nucleic acid amplification reaction according to a second embodiment of the present technology;

FIG. 5 is a schematic cross-sectional diagram illustrating a structure of a reaction region 43c of a microchip 1c for a nucleic acid amplification reaction according to a third embodiment of the present technology;

FIG. 6 is a schematic top diagram illustrating a structure of a reaction region 43d of a microchip 1d for a nucleic acid amplification reaction according to a fifth embodiment of the present technology; and

FIG. 7 is a schematic top diagram illustrating structures of introduction channels 331 and 332 and a reaction region 43e of a microchip for a nucleic acid amplification reaction according to related art.

### DETAILED DESCRIPTION OF THE EMBODIMENTS

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted. Note that the embodiments to be described below are shown as typical embodiments of the present technology, and do not narrowly limit interpretation of the scope of the present technology. The description is given of the following order.

1. Structure of microchip for nucleic acid amplification reaction according to first embodiment of present technology

(1) Introduction section, introduction channel, and reagent accommodation region

(2) Reagent

(3) Particles

(4) Particle accommodation section

2. Structure of microchip for nucleic acid amplification reaction according to modification of first embodiment
3. Structure of microchip for nucleic acid amplification reaction according to second embodiment of present technology
4. Structure of microchip for nucleic acid amplification reaction according to third embodiment of present technology
5. Structure of microchip for nucleic acid amplification reaction according to fourth embodiment of present technology
6. Structure of microchip for nucleic acid amplification reaction according to fifth embodiment of present technology
7. Structure of microchip for nucleic acid amplification reaction according to related art

### 1. Structure of Microchip for Nucleic Acid Amplification Reaction According to First Embodiment of Present Technology

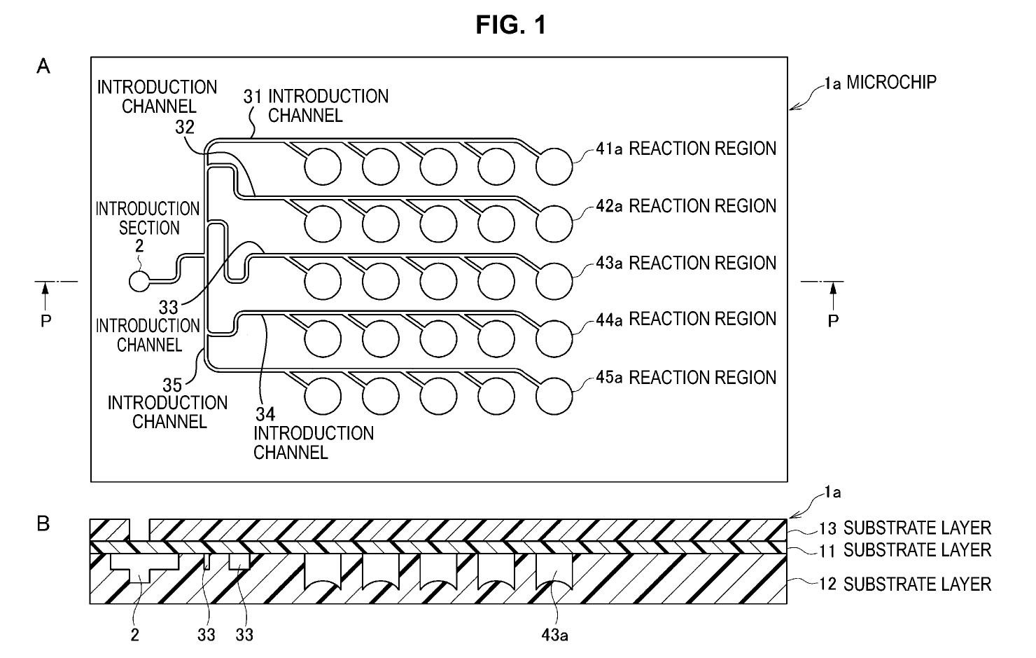

With reference to FIG. 1, a description is given of a structure of a microchip for a nucleic acid amplification reaction according to the first embodiment of the present technology.

### (1) Introduction Section, Introduction Channel, and Reagent Accommodation Region

FIG. 1A is a top diagram of a microchip for a nucleic acid amplification reaction (hereinafter, also referred to as a “microchip”) according to the first embodiment of the present technology denoted with reference numeral 1a in the figure. FIG. 1B is a schematic cross-sectional diagram corresponding to a cross section taken along the P-P line of FIG. 1A. The microchip 1a is provided with an introduction section 2 and reagent accommodation regions, the introduction section 2 being provided for introducing a sample containing a nucleic acid into the microchip 1a, the reagent accommodation regions each having particles and solid-phase reagents containing substances necessary for a nucleic acid amplification reaction. Since each “reagent accommodation region” accommodating the reagents and the particles is a reaction site of the nucleic acid amplification reaction in the present embodiment, the “reagent accommodation region” is referred to as a “reaction region” in the description of the embodiment. Since the “reagent accommodation region” provided in a microchip serves as a reaction site of a nucleic acid amplification reaction also in a second, third, and fourth embodiments to be described later, the “reagent accommodation region” is also referred to as the “reaction region” in the descriptions of the embodiments.

As illustrated in FIG. 1A, reaction regions 41a, 42a, 43a, 44a, and 45a each including the reagents and the particles are connected to introduction channels 31, 32, 33, 34, and 35, respectively. The sample introduced into the introduction section 2 flows through the introduction channels 31 to 35 and reaches the reaction regions 41a to 45a. Note that in FIG. 1 and in the description given with reference to FIG. 1, five reaction regions where the sample is supplied from the introduction channel 31 supplies are collectively referred to as the reaction region 41a, and likewise five reaction regions where the sample is supplied from each of the introduction channels 32, 33, 34, and 35 are the reaction region 42a, 43a, 44a, and 45a.

The sample introduced into the reaction regions 41a to 45a is a solution containing an analyte or a substance which generates an analyte by reacting with another substance. As the analyte, a nucleic acid such as DNA or RNA can be cited. A biological sample itself containing the analyte, such as blood, or a dilution thereof may also be used as the sample to be introduced into the reaction regions 41a to 45a.

As illustrated in FIG. 1B, the microchip 1a includes three substrate layers 11, 12, and 13 (the reagents and the particles are not illustrated in FIG. 1B). The substrate layers 11, 12, and 13 may be made of glass or a variety of plastics. The substrate layers 11, 12, and 13 may be bonded to each other by a publicly known technique such as: bonding using heat seal, an adhesive, anodic bonding, or an adhesive sheet; plasma activation bonding; or ultrasonic bonding. Note that when the substance held in the reaction regions 41a to 45a of the microchip 1a is optically analyzed, it is preferable that a material having a small optical error due to its optical transparency, less autofluorescence, and a small chromatic dispersion should be selected for the substrate layers 11, 12, and 13.

The substrate layer 12 is provided with the introduction section 2, the introduction channels 31 to 35, and the reaction regions 41a to 45a. To form the introduction section 2 and the like in the substrate layer 12, a publicly known technique may be used: for example, wet etching or dry etching of a glass substrate layer; or naoninprint, injection molding, or cutting of a plastic substrate layer. The introduction section 2 and the like may also be formed in the substrate layer 11. Part of the introduction section 2 and the like may be formed in the substrate layer 11, and the other part may be formed in the substrate layer 12. A substrate where the introduction section 2, the introduction channels 31 to 35, and the like are formed is not particularly limited.

### (2) Reagent

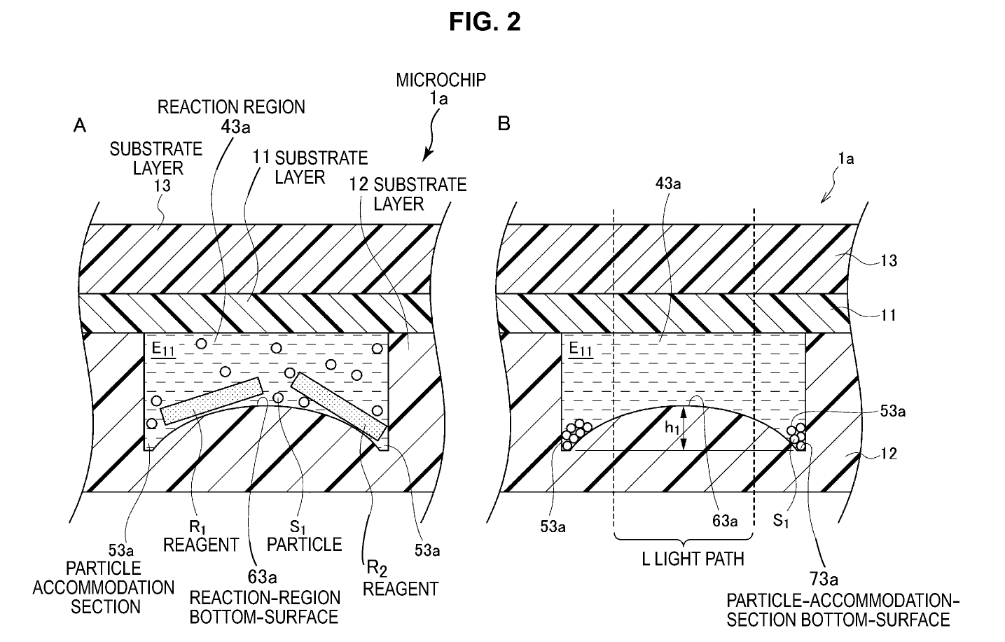

Next, reagents R₁ and R₂ included in the reaction regions 41a to 45a of the microchip 1a will be described with reference to FIG. 2A. FIG. 2 illustrates only the reaction region 43a as a representative of the reaction regions 41a to 45a provided in the microchip 1a.

FIG. 2A illustrates a state where the sample is introduced into a space E₁₁ in the reaction region 43a and where the reagents R₁ and R₂ are started to be dissolved. The reaction region 43a accommodates the solid-phase reagents R₁ and R₂. The reagents R₁ and R₂ contain at least part of substances necessary for obtaining amplified nucleic acid strands in the nucleic acid amplification reaction. Specifically, the reagents R₁ and R₂ contain an oligonucleotide primer complementary to at least part of an amplification target base sequence such as DNA or RNA, nucleic acid monomers (dNTPs), an enzyme, or an element contained in a reaction buffer solution, or the like. Although being not directly necessary for the nucleic acid amplification reaction, a probe labeled with fluorescent labeling or the like for detecting amplified nucleic acid strands: a detection reagent for intercalation into a double stranded nucleic acid; or the like may be an element included in the reagents R₁ and R₂ as the substance necessary for detecting the amplified nucleic acid strands.

Note that the “nucleic acid amplification reaction” using the microchip 1a includes: a PCR (Polymerase Chain Reaction) method which is related art and performs a temperature cycle; and a variety of isothermal amplification methods involving no temperature cycle. Examples of the isothermal amplification methods include an LAMP (Loop-Mediated Isothermal Amplification) method and a TRC (transcription-reverse transcription concerted) method. Besides, the “nucleic acid amplification reaction” widely includes nucleic acid amplification reactions for amplifying nucleic acids at an alternating temperature or at a constant temperature. Moreover, the “nucleic acid amplification reaction” includes reactions involving quantification of amplified nucleic acid strands such as a real time PCR method.

Although FIG. 2A illustrates an example in which the reaction region 43a accommodates two types of reagents (the reagents R₁ and R₂), the number or types of the reagents accommodated in the reaction regions 41a to 45a of the microchip 1a are not particularly limited. The type thereof may be one or more than one types. In addition, the reagents R₁ and R₂ having different compositions may be accommodated in the reaction regions 41a to 45a, or a plurality of reagents R₁ having the same composition may be included in the one reaction region 43a.

The reagents R₁ and R₂ included in the microchip 1a may be provided in such a manner that liquid or gelatinous reagents prepared to have predetermined compositions are solid phased in separate containers, and thereafter are accommodated in the reaction region 43a. Alternatively, liquid or gelatinous reagents are directly introduced into the reaction region 43a and are solid phased in the reaction region 43a, and the reagents anchored in the reaction region 43a may be used as the reagents R₁ and R₂.

### (3) Particles

The reaction region 43a includes particles S₁ in addition to the reagents R₁ and R₂ (see FIG. 2A). The number of the particles S₁ is not limited to the number illustrated in FIG. 2, and may be singular or plural. The size of the particles S₁ only have to be a size which can be used for mixing the reagents R₁ and R₂ with the sample in the reaction regions 41a to 45a and which allows the particles S₁ to be accommodated in a particle accommodation section 53a to be described later. The size thereof is not particularly limited.

When the particle accommodation section 53a is a recessed portion in a bottom surface of the reaction region 43a (reagent accommodation region), the particles S₁ preferably have a higher specific gravity than the sample introduced into the reaction region 43a (reagent accommodation region). Examples of the particles S₁ having the higher specific gravity than the sample include: particles of plastics such as polystyrene (PS) and polymethyl methacrylate (PMMA); alumina beads; zirconia beads; particles containing a metal such as steel balls; silica beads; and glass beads.

The particles S₁ promote the mixing of the sample with the reagents R₁ and R₂ by moving around inside the reaction region 43a. For moving the particles S₁ in the reaction region 43a, an introduction pressure of the sample introduced into the reaction region 43a may be utilized. The particles S₁ may be moved, for example, in such a manner that a user holds the microchip 1a with his/her hand to invert and shake the microchip 1a.

As illustrated in FIG. 2A, the space E₁₁ in the reaction region 43a is filled with the introduced sample. When the particles S₁ move around inside the space E₁₁, the sample around the particles S₁ and the solid-phase reagents R₁ and R₂ in the sample are stirred up, so that the mixing the sample with the reagents R₁ and R₂ is promoted.

### (4) Particle Accommodation Section

As illustrated in FIG. 2B, the microchip 1a includes the recessed portion formed in a reaction-region (reagent-accommodation-region) bottom-surface 63a. The recessed portion is the particle accommodation section 53a, and can accommodate the particles S₁ used for stirring up the sample and the reagents R₁ and R₂.

FIG. 2B illustrates a state where the reagents R₁ and R₂ are dissolved in the sample and where the particles S₁ are accommodated in the particle accommodation section 53a. In the microchip 1a where the sample and the reagents R₁ and R₂ are fully mixed together, the nucleic acid amplification reaction is started in the reaction region 43a. When amplified nucleic acid strands in the reaction region 43a in the process or the end of the nucleic acid amplification reaction are analyzed, it is desirable that the particles S₁ present in the reaction region 43a should not hinder the analysis. In particular, when the amplified nucleic acid strands are optically analyzed, it is desirable that the particles S₁ should not be present on an optical path of light emitted to or from the amplified nucleic acid strands. In the microchip 1a, the particle accommodation section 53a is formed out of a light path L shown by a broken line in FIG. 2B, and thus the particles S₁ accommodated in the particle accommodation section 53a do not hinder the analysis.

Since the particles S₁ in the reaction region 43a have the higher specific gravity than the sample, the particles S₁ fall inside the sample due to the mass of the particles S₁ themselves after moving around inside the space E₁₁, and come into contact with the reaction-region bottom-surface 63a. Since the reaction-region bottom-surface 63a is formed as a curved surface bulging toward a surface facing the reaction-region bottom-surface 63a, the particles S₁ reaching the reaction-region bottom-surface 63a move along the reaction-region bottom-surface 63a toward a peripheral portion of the reaction-region bottom-surface 63a and gather in the particle accommodation section 53a. Since the particle accommodation section 53a is a space formed as the recessed portion, the particles S₁ accommodated in the particle accommodation section 53a are locked in the particle accommodation section 53a until moving force is again applied to the particles S₁ by the shaking or the like of the microchip 1a performed by the user.

The particle accommodation section 53a may be provided in the microchip 1a at any position, as long as the position is out of an optical path of light used for analyzing nucleic acid strands in the reaction region 43a. The position is not particularly limited. In addition, a height h₁ of the reaction-region bottom-surface 63a with respect to a particle-accommodation-section bottom-surface 73a may be determined, based on the size and the number of the particles S₁, to have a height allowing the particles S₁ to be accommodated in the particle accommodation section 53a.

In the microchip 1a, the reaction regions 41a to 45a (reagent accommodation sections) include the particles S₁ as well as the solid-phase reagents R₁ and R₂. Accordingly, the mixing of the sample introduced into the microchip 1a with the reagents R₁ and R₂ is promoted, and thus the reagents R₁ and R₂ are fully dissolved in the sample, so that it is less likely to have variation in degree of the reagents R₁ and R₂ dissolution among the reaction regions 41a to 45a. Moreover, the accommodation of the reagents R₁ and R₂ in the reaction regions 41a to 45a reduces loss of the reagents R₁ and R₂ in comparison with the case where the reagents R₁ and R₂ are dissolved in a separately provided space in the microchip 1a and thereafter introduced into the reaction regions 41a to 45a. Accordingly, time taken to dissolve the reagents R₁ and R₂ is made shorter in the analysis using the microchip 1a. In addition, it is less likely to have variation among the reaction regions 41a to 45a in the nucleic acid amplification reaction, and thus it is possible to perform a higher-accuracy analysis.

Besides, the microchip 1a includes the reaction regions 41a to 45a each provided with the particle accommodation section 53a accommodating the particles S₁. This makes it possible to gather the particles S₁ at a predetermined position where the analysis of the amplified nucleic acid strands in each of the reaction regions 41a to 45a is not hindered. Since the particles S₁ do not hinder the analysis, the accuracy of the analysis using the microchip 1a is enhanced.

It is preferable to use: an elastic material for the substrate layer 11 included in the microchip 1a; and a gas impermeable material for the substrate layers 12 and 13. Since the substrate layer 11 is made of the elastic material, the sample can be introduced into the introduction section 2, of the microchip 1a, sealed by the substrate layer 11, by using a needle member such as a needle. The sample is introduced into the microchip 1a in the following manner, for example. A syringe or the like filled with the sample is connected with the needle member, and the tip end of the needle member is made to penetrate the substrate layer 11 from the outside of the microchip 1a to reach the introduction section 2, so that the inside of the syringe is connected with the introduction section 2.

As the elastic material of the substrate layer 11, a silicone-based elastomer such as polydimethylsiloxane (PDMS), an acrylic-based elastomer, a urethane-based elastomer, a fluorine-based elastomer, a styrene-based elastomer, an epoxy-based elastomer, a natural rubber, and the like can be cited.

In contrast, the substrate layers 12 and 13 are made of the gas-impermeable material, and thereby it is possible to prevent the sample introduced into the reaction regions 41a to 45a from being gasified due to heating or the like, permeating through the substrate layer 11, and then disappearing (liquid loss).

As the gas-impermeable material of the substrate layers, glass, plastics, metals, ceramics, and the like can be cited. As the plastics, PMMA (polymethyl methacrylate: an acrylic resin), PC (polycarbonate), and the like can be cited. As the metals, aluminum, copper, stainless (SUS), silicon, titan, tungsten, and the like can be cited. As the ceramics, alumina (Al₂O₃), aluminum nitride (AlN), silicon carbide (SiC), titanium oxide (TiO₂), zirconia oxide (ZrO₂), quartz, and the like can be cited.

In the microchip 1a, regions such as the introduction section 2 and the reaction regions 41a to 45a into which the sample is introduced are preferably set at a negative pressure (for example, 1/100 of an atmospheric pressure) with respect to an atmospheric pressure. When the sample is introduced into the microchip 1a by using the needle member, setting the inside of the microchip 1a at the negative pressure with respect to the atmospheric pressure makes it possible to automatically suck the sample in the syringe into the microchip 1a through the needle member due to a pressure difference between the outside (the inside of the syringe) and the inside of the microchip 1a. What is necessary for setting the inside of the microchip 1a at the negative pressure with respect to the atmospheric pressure is to use the negative pressure in bonding the substrate layer 11 to the substrate layer 12, for example.

### 2. Structure of Microchip for Nucleic Acid Amplification Reaction According to Modification of First Embodiment

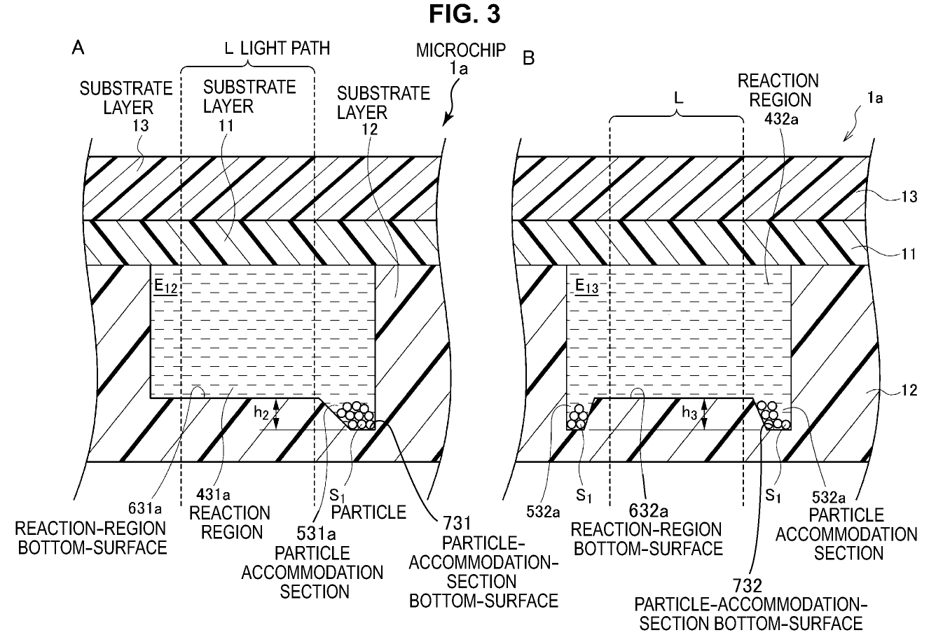

FIG. 3 illustrates a reaction region 431a and a reaction region 432a as representatives of reaction regions of a microchip for a nucleic acid amplification reaction according to a modification of the first embodiment. FIGS. 3A and 3B each illustrate a state where the reagents R₁ and R₂ are dissolved in the sample introduced in a space E₁₂ or E₁₃, and the particles S₁ are accommodated in a particle accommodation section 531a or 532a.

The modification of the first embodiment uses the same structure as that in the first embodiment except in the structure of reaction regions such as the reaction regions 431a and 432a. Structural elements that have the same structure as that in the first embodiment are denoted with the same reference numerals, and repeated explanation thereof is omitted. The substrate layers 11, 12, and 13 in the modification of the first embodiment are respectively made of the same materials as those of the substrate layers denoted with the same reference numerals in the microchip 1a.

The reaction region 431a illustrated in FIG. 3A includes the particles S₁, and the particle accommodation section 531a is provided out of the light path L. A bottom surface of a reaction region does not necessarily have to be formed as a curved surface in the microchip 1a according to the first embodiment of the present technology. As illustrated in FIG. 3A, a reaction-region bottom-surface 631a may be flat.

Since the reaction-region bottom-surface 631a is flat, the particles S₁ reach the reaction-region bottom-surface 631a due to the mass of the particles S₁, and thereafter stay at the fall position on the reaction-region bottom-surface 631a. Then, for example, the user tilts the microchip 1a, and thereby the particles S₁ on the reaction-region bottom-surface 631a move and gather in the particle accommodation section 531a.

As illustrated in FIG. 3B, the position where a particle accommodation section 532a is formed is not limited to one side of the reaction region 432a. For example, the particle accommodation section 532a may be provided on each side of the light path L (see broken lines in FIG. 3B), or may be provided along an inner periphery of the reaction region 432a around the light path L. The positions where the particle accommodation sections 531a and 532a are formed are not particularly limited. For example, when amplified nucleic acid strands are optically analyzed, the particle accommodation sections 531a and 532a only have to be formed out of a region (the light paths L in FIGS. 3A and 3B) where the analysis is hindered.

In addition, in the microchip 1a according to the first embodiment, the particle accommodation sections 531a and 532a only have to be the recessed portions of the reaction-region bottom-surface 631a and a reaction-region bottom-surface 632a, respectively, and particle-accommodation-section bottom-surfaces 731 and 732 only have to be located below the reaction-region bottom-surfaces 631a and 632a. Heights h₂ and h₃ of the respective reaction-region bottom-surfaces 631a and 632a with respect to the particle-accommodation-section bottom-surfaces 731 and 732 may be determined, based on the size and the number of the particles S₁, to have heights allowing the particles S₁ to be accommodated in the particle accommodation sections 531a and 532a.

### 3. Structure of Microchip for Nucleic Acid Amplification Reaction According to Second Embodiment of Present Technology

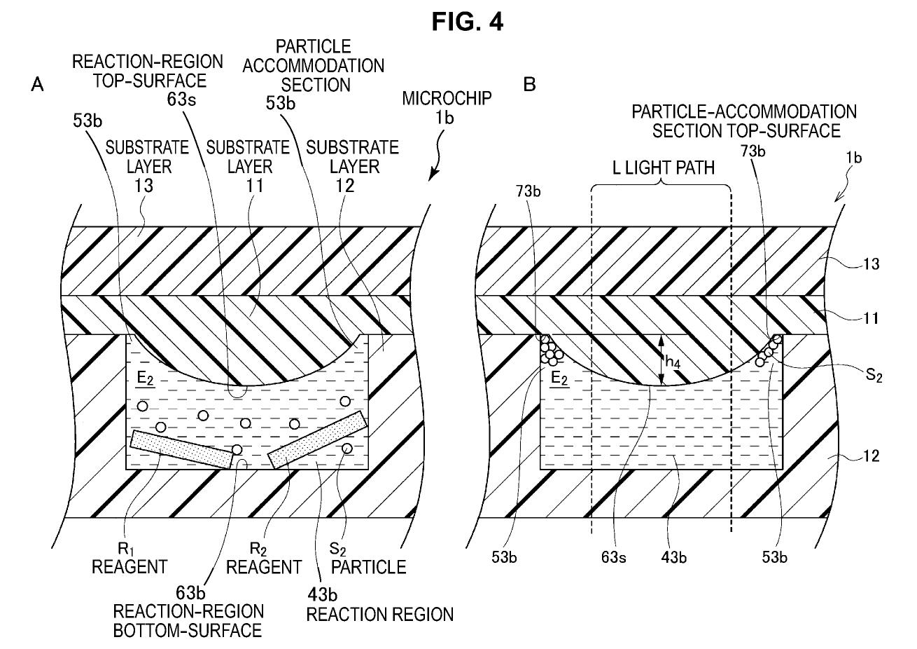

FIG. 4 illustrates a schematic cross-sectional diagram of a reaction region 43b of a microchip 1b according to a second embodiment of the present technology, the reaction region 43b representing reaction regions 41b, 42b, 44b, and 45b of the microchip 1b. FIG. 4A illustrates a state where the sample is introduced into a space E₂ in the reaction region 43b and where the reagents R₁ and R₂ are started to be dissolved. FIG. 4B illustrates a state where the reagents R₁ and R₂ are dissolved in the sample and where particles S₂ are accommodated in a particle accommodation section 53b.

The microchip 1b has the same structure as that in the first embodiment except the reaction regions 41b to 45b, the particles S₂ accommodated in the reaction regions 41b to 45b, and the particle accommodation section 53b. Structural elements that have the same structure as that in the first embodiment are denoted with the same reference numerals, and repeated explanation thereof is omitted. The substrate layers 11, 12, and 13 included in the microchip 1b are respectively made of the same materials as those of the substrate layers denoted with the same reference numerals in the microchip 1a.

The reaction region 43b of the microchip 1b includes the reagents R₁ and R₂ and the particles S₂ (see FIG. 4A). Since the particle accommodation section 53b to be described later is located above a reaction-region top-surface 63s, the particles S₂ preferably have a lower specific gravity than the sample has. Examples of a material of the particles S₂ having the lower specific gravity include a variety of plastics such as polyethylene (PE), polypropylene (PP), and polymethylpentene (PMP).

Like the particles S₁ included in the microchip 1a according to the first embodiment, the particles S₂ included in the reaction region 43b move around inside the reaction region 43b due to the introduction pressure of the sample introduced into the reaction region 43b and inverting and shaking of the microchip 1b by the user, and thereby the sample and the reagents R₁ and R₂ in the sample are stirred up. Thus, the mixing of the sample with the reagents R₁ and R₂ is promoted.

As illustrated in FIG. 4B, the microchip 1b includes a recessed portion formed in a surface facing a reaction-region (reagent-accommodation-region) bottom-surface 63b. The recessed portion is the particle accommodation section 53b, and the particles S₂ can be accommodated in a space formed as the recessed portion.

The particles S₂ have the lower specific gravity than the sample has, and thus float in the sample to reach the reaction-region top-surface 63s. The reaction-region top-surface 63s which is a surface facing a bottom surface of the reaction region (reagent accommodation region) 43b is formed as a curved surface bulging toward the reaction-region bottom-surface 63b. For this reason, the particles S₂ reaching the reaction-region top-surface 63s move along a peripheral edge portion of the reaction-region top-surface 63s and gather in the particle accommodation section 53b. Since the particle accommodation section 53b is the space formed as the recessed portion, the particles S₂ accommodated in the particle accommodation section 53b are locked in the particle accommodation section 53b until moving force is again applied to the particles S₂ by the shaking or the like of the microchip 1a performed by the user.

The reaction-region top-surface 63s does not necessarily have to be formed as the curved surface in the microchip 1b according to the second embodiment of the present technology. Like the reaction-region bottom-surfaces 631a and 632a according to the modification of the first embodiment which are illustrated in FIGS. 3A and 3B, the reaction-region top-surface 63s with which the particles S₂ come in contact may be flat. When the reaction-region top-surface 63s is flat, for example, the user may tilt the microchip 1b to move the particles S₂ to be accommodated in the particle accommodation section 53b.

In the microchip 1b, the particle accommodation section 53b only has to be the recessed portion of the reaction-region top-surface 63s, and a particle-accommodation section top-surface 73b which is a bottom of the recessed portion only has to be located above the reaction-region top-surface 63s. A height h₄ of the particle-accommodation section top-surface 73b with respect to the reaction-region top-surface 63s only has to be a height allowing the particles S₂ to be accommodated in the particle accommodation section 53b, according to the size and the number of the particles S₂.

The particle accommodation section 53b in the reaction region 43b may be provided at any position, as long as the analysis of the amplified nucleic acid strands is not hindered at the position. For example, when the amplified nucleic acid strands are optically analyzed, the particle accommodation section 53b is preferably provided out of the light path L shown by broken lines in FIG. 4B.

Like the microchip 1a according to the first embodiment, the microchip 1b includes the particles S₂ in the region where the reagents R₁ and R₂ are accommodated. Thereby, the sample and the reagents R₁ and R₂ in the sample are stirred up due to the particles S₂, so that the mixing of the sample with the reagents R₁ and R₂ is promoted. For this reason, the reagents R₁ and R₂ are fully dissolved, there is less variation in sample among the reaction regions 41b to 45b, and thus it is possible to perform a higher-accuracy analysis by using the microchip 1b.

In addition, the microchip 1b includes the particle accommodation section 53b accommodating the particles S₂ which is provided in the reaction region 43b. Thereby, the particles S₂ used for stirring the sample are placed at a predetermined position in analyzing the amplified nucleic acid strands, so that the analysis is prevented from being hindered. This leads to enhancement of the analysis accuracy using the microchip 1b.

### 4. Structure of Microchip for Nucleic Acid Amplification Reaction According to Third Embodiment of Present Technology

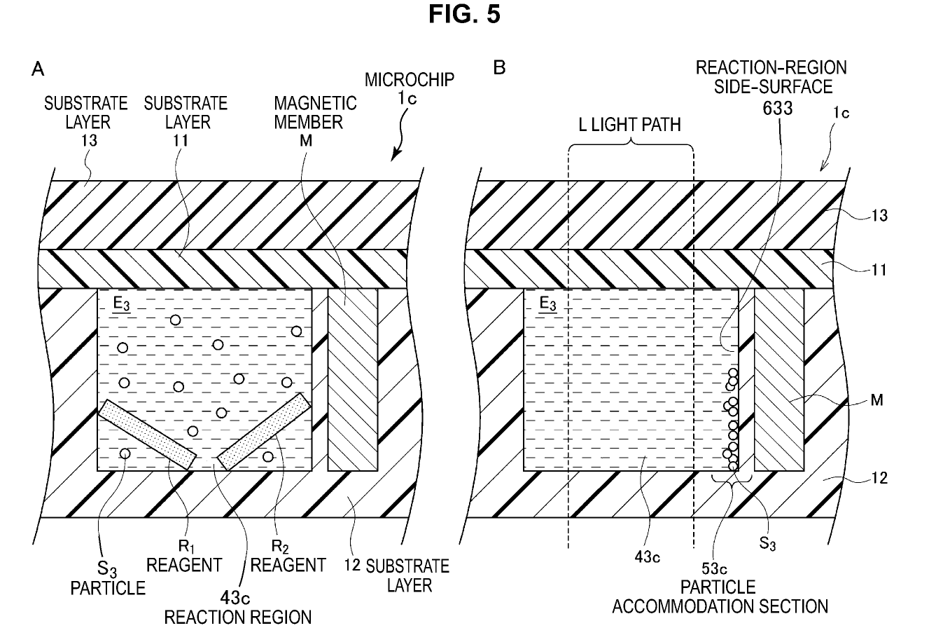

FIG. 5 illustrates a schematic cross-sectional diagram of a reaction region 43c of a microchip 1c according to a third embodiment of the present technology, the reaction region 43c representing reaction regions 41c, 42c, 44c, and 45c of the microchip 1c. FIG. 5A illustrates a state where the sample is introduced into a space E₃ in the reaction region 43c and where the reagents R₁ and R₂ are started to be dissolved. FIG. 5B illustrates a state where the reagents R₁ and R₂ are dissolved in the sample and where particles S₃ are trapped in a particle accommodation section 53c.

The microchip 1c has the same structure as that in the first embodiment except the reaction regions 41c to 45c, the particles S₃ accommodated in the reaction regions 41c to 45c, and a magnetic member M. Structural elements that have the same structure as that in the first embodiment are denoted with the same reference numerals, and repeated explanation thereof is omitted. The substrate layers 11, 12, and 13 included in the microchip 1c are respectively made of the same materials as those of the substrate layers denoted with the same reference numerals in the microchip 1a.

When the magnetic member M to be described later is provided near the reaction region 43c of the microchip 1c, the particles S₃ included in the reaction region 43c are preferably magnetic. As the particles S₃, commercially available magnetic latex particles and magnetic silica particles may be used.

As in the case of the microchip 1a in the first embodiment, the particles S₃ in the reaction region 43c move around inside the space E₃ in the reaction region 43c, and thereby the sample and the reagents R₁ and R₂ in the sample are stirred up. Thus, the mixing of the sample with the reagents R₁ and R₂ is promoted (see FIG. 5A).

In the microchip 1c, the magnetic member M is provided at a position from which magnetic force is exerted on a predetermined space corresponding to the particle accommodation section 53c. For this reason, the particles S₃ gather in the particle accommodation section 53c due to the magnetic force of the magnetic member M, and are trapped in the particle accommodation section 53c. FIG. 5B illustrates an example of providing the magnetic member M near a reaction-region side-surface 633. The particles S₃ are attracted to the reaction-region side-surface 633 due to the magnetic force of the magnetic member M and reach the reaction-region side-surface 633.

The magnetic member M may be made of an electromagnet or a permanent magnet. When the electromagnet is used, it is possible to gather the particles S₃ in the particle accommodation section 53c by generating a magnetic field in such a manner that a current is applied to a coil after the sample is stirred with the particles S₃. When the permanent magnet is used, the magnetic member M may be provided to obtain magnetic force which enables the particles S₃ to move around inside the reaction region 43c utilizing a pressure for introducing the sample into the reaction region 43c or force equivalent to an action of the user to shake the microchip 1c. Note that the magnetic member M does not necessarily have to be included in the microchip 1c, and may be provided outside the microchip 1c.

That is, the space in the reaction region 43c on which the magnetic force of the magnetic member M is exerted is the particle accommodation section 53c. The position of the particle accommodation section 53c in the reaction region 43c is not limited to the position illustrated in FIG. 5B at which the particle accommodation section 53c is on the reaction-region side-surface 633, and may be any position, as long as an analysis in the reaction region 43c is not hindered at the position. For example, when the amplified nucleic acid strands in the reaction region 43c are optically analyzed, the particle accommodation section 53c is preferably provided out of the light path L as illustrated in FIG. 5B.

In the microchip 1c, the magnetic member M causes the particles S₃ to be trapped in the reaction region 43c at the predetermined position. This prevents the particles S₃ from hindering the analysis of the amplified nucleic acid strands and thus enables a higher accuracy analysis using the microchip 1c. In addition, it is not necessary to form the recessed portion in the reaction region 43c, and thus it is possible to easily form the substrate layers 11, 12, and 13 included in the microchip 1c. Except the structure and advantageous effects described above, the microchip 1c has the same structure and advantageous effects as those of the microchips 1a and 1b according to the first and second embodiments.

### 5. Structure of Microchip for Nucleic Acid Amplification Reaction According to Fourth Embodiment of Present Technology

Particles included in reaction regions (reagent accommodation regions) of a microchip according to the present embodiment are preferably thermally melted when a nucleic acid amplification reaction using the microchip involves a heating operation.

Like the particles S₁ to S₃ in the first embodiment and the like, thermally melting particles included in each reaction region of the microchip according to the present embodiment cause the sample and the reagents R₁ and R₂ to be stirred up in the reaction region, so that the mixing of the sample with the reagents R₁ and R₂ is promoted. After the dissolution of the reagents R₁ and R₂ is completed, the microchip is heated for the nucleic acid amplification reaction. The heating operation causes the thermally melting particles in the reaction region to be dissolved, and the analysis of the amplified nucleic acid strands is prevented from being hindered due to the particles.

The thermally melting material is not particularly limited, as long as the material is a material which does not hinder the nucleic acid amplification reaction when the material is dissolved, and is a material which is dissolved at a heating temperature used for the nucleic acid amplification reaction. Examples of the thermally melting particles include low-melting paraffin, eicosane, and grease.

In the microchip according to the present embodiment, the particles are thermally melted, and thus do not hinder the analysis of the amplified nucleic acid strands. In addition, no necessity for forming a recessed portion in the reaction region and for providing a magnetic member leads to simplified structure of the microchip and easy manufacturing thereof. Except the structure and advantageous effects described above, the present embodiment has the same structure and advantageous effects as those of the microchips 1a and 1b according to the first and second embodiments.

### 6. Structure of Microchip for Nucleic Acid Amplification Reaction According to Fifth Embodiment of Present Technology

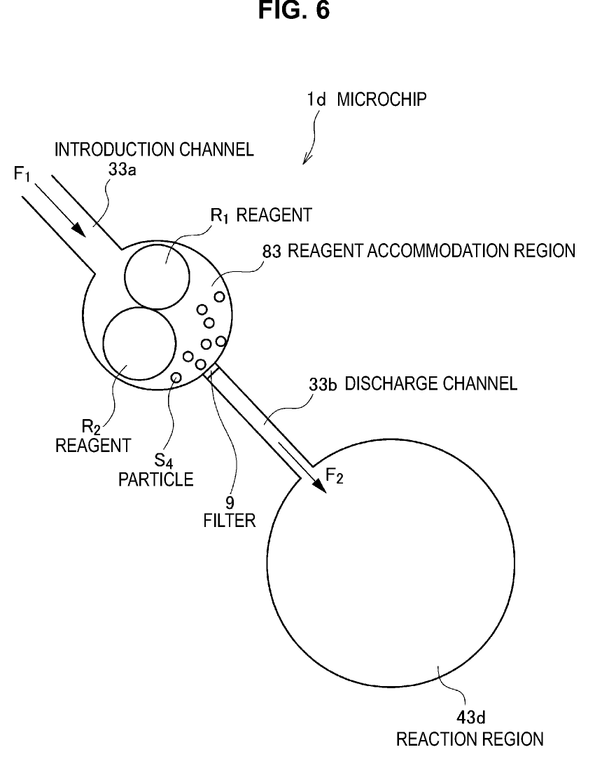

FIG. 6 illustrates a schematic top diagram of a reaction region 43d of a microchip 1d according to a fifth embodiment of the present technology, the reaction region 43d representing reaction regions 41d, 42d, 44d, and 45d of the microchip 1d. The microchip 1d has the same structure as that in the first embodiment except the reaction regions 41d to 45d, a reagent accommodation region 83 where the reagents R₁ and R₂ are accommodated, and a discharge channel 33b connecting the reaction regions 41d to 45d and the reagent accommodation region 83. Structural elements that have the same structure as that in the first embodiment are denoted with the same reference numerals, and repeated explanation thereof is omitted. The substrate layers 11, 12, and 13 included in the microchip 1d are respectively made of the same materials as those of the substrate layers denoted with the same reference numerals in the microchip 1a.

The microchip 1d illustrated in FIG. 6 is provided with the reaction region 43d which is a reaction site of the nucleic acid amplification reaction, separately from the reagent accommodation region 83 including particles S₄ and the reagents R₁ and R₂. The reagent accommodation region 83 and the reaction region 43d are connected with each other by a discharge channel 33b. The reagent accommodation region 83 is also connected with the introduction section 2 by an introduction channel 33a (the introduction section 2 is not illustrated in FIG. 6).

In the present embodiment, the sample introduced into the introduction section 2 flows through the introduction channel 33a and reaches the reagent accommodation region 83 (see the arrow F₁ in FIG. 6). The sample is mixed with the reagents R₁ and R₂ in the reagent accommodation region 83. At this time, like the particles S₁ or the like in the aforementioned first embodiment, the particles S₄ included in the reagent accommodation region 83 move around inside the reagent accommodation region 83, and thereby the sample and the reagents R₁ and R₂ in the sample are stirred up, so that the mixing of the sample with the reagents R₁ and R₂ is promoted.

Larger sizes of the reagents R₁ and R₂ than a channel diameter of the discharge channel 33b prevent the reagents R₁ and R₂ from flowing through the discharge channel 33b until the reagents R₁ and R₂ are dissolved. The particles S₄ can also prevent the solid-phase reagents R₁ and R₂ from flowing through the discharge channel 33b. For this reason, after being dissolved in the sample, the reagents R₁ and R₂ are introduced into the reaction region 43d (see the arrow F₂ in FIG. 6).

Since a communication section between the reagent accommodation region 83 and the discharge channel 33b is provided with a filter 9, the particles S₄ stay in the reagent accommodation region 83. This prevents the particles S₄ from being introduced into the reaction region 43d to hinder the analysis in the reaction region 43d. When the filter 9 is not provided, what is necessary is to make the size of the particles S₄ larger than the channel diameter of the discharge channel 33b, like the reagents R₁ and R₂.

In the microchip 1d according to the present embodiment, the particles S₄ are included in the space other than the reaction region 43d. Accordingly, it is not necessary to provide a structure in which the particles S₄ are accommodated in the reaction region 43d. Since it is not necessary to consider an impact on the analysis in the reaction region 43d, the material of the particles S₄ included in the microchip 1d is not particularly limited. Except the structure and advantageous effects described above, the present embodiment has the same structure and advantageous effects as those of the microchips 1a and 1b according to the first and second embodiments.

### 7. Structure of Microchip for Nucleic Acid Amplification Reaction According to Related Art

In the microchip according to each embodiment of the present technology, for promoting the mixing of the sample introduced into the microchip with the solid-phase reagents, connections may be made between the introduction channels and the reaction regions, or between the introduction channels and the reagent accommodation regions so that the sample can circulate in each reaction region or each reagent accommodation region.

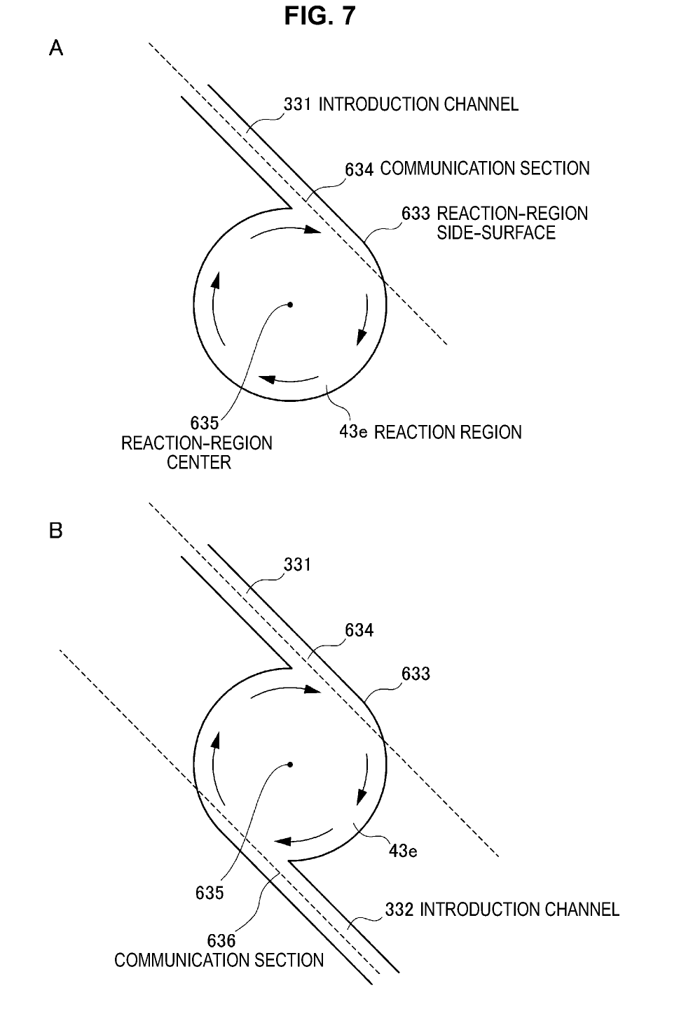

FIG. 7A illustrates a reaction region 43e and an introduction channel 331 which are provided in a microchip (the reagents are not illustrated in FIG. 7A), as an example of the aforementioned structure. The introduction channel 331 extends in a direction of a tangent of a circumferential surface of the reaction region 43e which has a substantially circular shape in a top view (see a broken line in FIG. 7A). For this reason, the sample flowing through the introduction channel 331 to be introduced into the reaction region 43e swirls along a substantially circular reaction-region side-surface 633, so that a swirl flow of the sample is generated (see the arrows in FIG. 7A).

The position of a communication section 634 which is a connection section between the introduction channel 331 and the reaction region 43e is not limited to a position shown in FIG. 7, and only has to be a position enabling the generation of the swirl flow of the sample in the reaction region 43e. It is preferable that a line extended from the introduction channel 331 (see a broken line in FIG. 7A) should not pass through the center of the reaction region 43e (a reaction-region center 635).

In addition, to easily generate the swirl flow of the sample caused by the introduction into the reaction region 43e, two introduction channels of the introduction channel 331 and an introduction channel 332 may be connected to the one reaction region 43e as illustrated in FIG. 7B, for example. The number of the introduction channels connected to the reaction region 43e is not particularly limited. Also when the two introduction channels 331 and 332 are connected to the reaction region 43e, the introduction channels 331 and 332 preferably extend in the direction of the tangent of the circumferential surface included in the reaction region 43e (see broken lines in FIG. 7B). The sample is introduced from the communication section 634 and a communication section 636 of the introduction channels 331 and 332 into the reaction region 43e, so that the swirl flow of the sample is generated (see the arrows in FIG. 7B).

Although the shape of the reaction region provided in the microchip according to the embodiment of the present technology is not limited to the substantially circle in the top view, the reaction region 43e preferably have the circular shape in the top view to easily generate the swirl flow of the sample in the reaction region 43e.

In addition, the generation of the swirl flow of the sample is not limited to the generation in the reaction region 43e illustrated in FIG. 7. When the reagents R₁ and R₂ are included in the reagent accommodation region 83 (see FIG. 6) as in the microchip 1d in the fifth embodiment, it is also possible to form the introduction channel 33a for causing the sample to flow through the reagent accommodation region 83 so that the swirl flow of the sample can be generated.

With the aforementioned structure of the introduction channels 331 and 332 connected to the reaction region 43e, the swirl flow is generated in the sample introduced into the reaction region 43e, so that the mixing of the sample with the reagents R₁ and R₂ is promoted. When the particles are included in the microchip as in the reaction region of each of the first to fifth embodiments, the generation of the swirl flow of the sample causes the particles to move more dynamically, so that the mixing of the sample with the reagents R₁ and R₂ due to the particles is further promoted.

It should be understood by those skilled in the art that various modifications, combinations, sub-combinations and alterations may occur depending on design requirements and other factors insofar as they are within the scope of the appended claims or the equivalents thereof.

Additionally, the present technology may also be configured as below.

(1) A microchip for a nucleic acid amplification reaction including

a reagent accommodation region including a solid-phase reagent and a particle, the solid-phase reagent containing a substance necessary for a nucleic acid amplification reaction.

(2) The microchip for a nucleic acid amplification reaction according to (1),

wherein a particle accommodation section allowing the particle to be accommodated therein is provided in the reagent accommodation region at a predetermined position.

(3) The microchip for a nucleic acid amplification reaction according to (2),

wherein the particle has a higher specific gravity than a sample to be introduced into the reagent accommodation region.

(4) The microchip for a nucleic acid amplification reaction according to (3),

wherein the particle accommodation section is a recessed portion of a bottom surface of the reagent accommodation region.

(5) The microchip for a nucleic acid amplification reaction according to (4),

wherein the bottom surface of the reagent accommodation region is formed as a curved surface bulging toward a surface facing the bottom surface.

(6) The microchip for a nucleic acid amplification reaction according to (2),

wherein the particle has a lower specific gravity than a sample to be introduced into the reagent accommodation region.

(7) The microchip for a nucleic acid amplification reaction according to (6),

wherein the particle accommodation section is a recessed portion of a surface facing a bottom surface of the reagent accommodation region.

(8) The microchip for a nucleic acid amplification reaction according to (7),

wherein the surface facing the bottom surface of the reagent accommodation region is formed as a curved surface bulging toward the bottom surface.

(9) The microchip for a nucleic acid amplification reaction according to (1) or (2),

wherein the particle is magnetic.

(10) The microchip for a nucleic acid amplification reaction according to (9), wherein a magnetic member is provided at a position from which magnetic force is exerted on the particle accommodation section.
(11) The microchip for a nucleic acid amplification reaction according to (1),

wherein the particle is thermally melted.

(12) The microchip for a nucleic acid amplification reaction according to any one of (1) to (11),

wherein the reagent accommodation region is a reaction site of the nucleic acid amplification reaction.

(13) The microchip for a nucleic acid amplification reaction according to (1), including

a reaction site for the nucleic acid amplification reaction which is connected with the reagent accommodation region by a flow channel.

(14) The microchip for a nucleic acid amplification reaction according to (13),

wherein the reagent accommodation region is connected with an introduction section for a sample by a flow channel.

According to the microchip for nucleic acid amplification according to the embodiments of the present technology, the sample introduced into the microchip can be mixed with the solid-phase reagents in a simple manner. Thus, the microchip for a nucleic acid amplification reaction according to the embodiments of the present technology is usable for a nucleic acid test for genotyping, pathogen characterization, or the like in clinical practice.

The present disclosure contains subject matter related to that disclosed in Japanese Priority Patent Application JP 2012-174415 filed in the Japan Patent Office on Aug. 6, 2012, the entire content of which is hereby incorporated by reference.

What is claimed is:

1. A microchip for a nucleic acid amplification reaction comprising
a reagent accommodation region including a solid-phase reagent and a particle, the solid-phase reagent containing a substance necessary for a nucleic acid amplification reaction.

2. The microchip for a nucleic acid amplification reaction according to claim 1,
wherein a particle accommodation section allowing the particle to be accommodated therein is provided in the reagent accommodation region at a predetermined position.

3. The microchip for a nucleic acid amplification reaction according to claim 2,
wherein the particle has a higher specific gravity than a sample to be introduced into the reagent accommodation region.

4. The microchip for a nucleic acid amplification reaction according to claim 3,
wherein the particle accommodation section is a recessed portion of a bottom surface of the reagent accommodation region.

5. The microchip for a nucleic acid amplification reaction according to claim 4,
wherein the bottom surface of the reagent accommodation region is formed as a curved surface bulging toward a surface facing the bottom surface.

6. The microchip for a nucleic acid amplification reaction according to claim 2,
wherein the particle has a lower specific gravity than a sample to be introduced into the reagent accommodation region.

7. The microchip for a nucleic acid amplification reaction according to claim 6,
wherein the particle accommodation section is a recessed portion of a surface facing a bottom surface of the reagent accommodation region.

8. The microchip for a nucleic acid amplification reaction according to claim 7,
wherein the surface facing the bottom surface of the reagent accommodation region is formed as a curved surface bulging toward the bottom surface.

9. The microchip for a nucleic acid amplification reaction according to claim 2,
wherein the particle is magnetic.

10. The microchip for a nucleic acid amplification reaction according to claim 9,
wherein a magnetic member is provided at a position from which magnetic force is exerted on the particle accommodation section.

11. The microchip for a nucleic acid amplification reaction according to claim 1,
wherein the particle is thermally melted.

12. The microchip for a nucleic acid amplification reaction according to claim 1,
wherein the reagent accommodation region is a reaction site of the nucleic acid amplification reaction.

13. The microchip for a nucleic acid amplification reaction according to claim 1, comprising
a reaction site for the nucleic acid amplification reaction which is connected with the reagent accommodation region by a flow channel.

14. The microchip for a nucleic acid amplification reaction according to claim 13,
wherein the reagent accommodation region is connected with an introduction section for a sample by a flow channel.
